(19) United States
(12) Patent Application Publication
(10) Pub. No.: US 2022/0347170 A1
(43) Pub. Date: Nov. 3, 2022

(54) Method of Reducing Brow Ache

(71) Applicant: Lenz Therapeutics, Inc., Rancho Santa Fe, CA (US)

(72) Inventor: Gerald Horn, Deerfield, IL (US)

(21) Appl. No.: 17/730,376

(22) Filed: Apr. 27, 2022

Related U.S. Application Data
(60) Provisional application No. 63/180,691, filed on Apr. 28, 2021.

(51) Int. Cl. A61K 31/4748; A61K 31/4178; A61K 47/26; A61P 29/00; A61K 47/38

(52) U.S. Cl. CPC A61K 31/4748; A61K 31/4178; A61K 47/26; A61P 29/00; A61K 47/38

(57) ABSTRACT

The present invention relates to methods of reducing or eliminating brow ache when administering an ophthalmic drug comprising formulating and administering the ophthalmic drug with a surfactant and a viscosity enhancer to create a composition having a viscosity of about 25 centipoise or less at a shear rate of 1/1000 per second at 25 degrees Celsius and a viscosity of about 70 centipoise at shear rate of 1 per second at 25 degrees Celsius. The present invention further relates to methods of treating an eye condition selected from the group consisting of presbyopia, myopia, hyperopia and astigmatism without inducing brow ache comprising administering a composition comprising a miotic agent, a surfactant and a viscosity enhancer, wherein the composition has a viscosity of about 25 centipoise or less at a shear rate of 1/1000 per second at 25 degrees Celsius and a viscosity of about 70 centipoise or more at shear rate of 1 per second at 25 degrees Celsius.

### FIELD OF THE INVENTION

The present invention relates to methods of reducing or eliminating brow ache when administering an ophthalmic drug comprising formulating the ophthalmic drug with a surfactant and a viscosity enhancer to create a composition having a viscosity of about 25 centipoise or less at a shear rate of 1/1000 per second at 25 degrees Celsius and a viscosity of about 70 centipoise at shear rate of 1 per second at 25 degrees Celsius and administering the composition to a subject in need thereof.

The present invention further relates to a method of treating an eye condition selected from the group consisting of presbyopia, myopia, hyperopia and astigmatism comprising administering a composition comprising a miotic agent, a surfactant and a viscosity enhancer to a subject in need thereof, wherein the composition has a viscosity of about 25 centipoise or less at a shear rate of 1/1000 per second at 25 degrees Celsius and a viscosity of about 70 centipoise or more, preferably about 150 centipoise or more and more preferably about 300 centipoise or more at shear rate of 1 per second at 25 degrees Celsius and wherein administration of the composition does not cause the subject to suffer from ciliary spasm induced brow ache or headache.

### BACKGROUND OF THE INVENTION

Brow ache can be induced by ciliary spasms often coincident with the instillation of an ophthalmic drug. The inducement of brow ache may lead to poor patient compliance and ineffective maintenance of the eye condition being treated. For example, miotic agents used to treat presbyopia often lead to unpleasant brow ache.

Presbyopia, loosely translated as “old-man eye” is a condition in which an individual minimum focal length continues to increase despite excellent unaided distance vision. Specifically, as a person ages the minimum distance from the eye at which an object will come into focus, provided distance vision is corrected or is excellent unaided, increases. For example, a 10 year-old can focus on an object or a “focal point” only three inches (0.072 meters) from their eye while still retaining excellent distance vision; a 40 year-old at six inches (0.15 meters); and a 60 year-old at an inconvenient 39 inches (1.0 meter).

The highest incidence of first complaint of presbyopia occurs in people ages 42-44. Presbyopia occurs because as a person ages the eye's accommodative ability which uses near reflex-pupil constriction, convergence of the eyes and particularly ciliary muscle contraction decreases. This reduction in accommodation results in an inadequate change in the normal thickening and increased curvature of the anterior surface of the lens that is necessary for the shift in focus from distant objects to near objects. Important near focus tasks affected by presbyopia include viewing computer screens (21 inches) and reading print (16 inches).

Presbyopia is a normal and inevitable effect of ageing and is the first unmistakable sign for many in their forties that they are getting older. One study found that more than 1 billion people worldwide were presbyopic in 2005. This same study predicted that number to almost double by the year 2050. If everyone over the age of 45 is considered to be presbyopic, then an estimated 122 million people in the United States alone had presbyopia in 2010. As baby boomers reach the critical age, this number is only going to increase.

Presbyopia carries with it a stigma resulting from the limitation in ability to quickly function at many tasks requiring focusing at both distant and near points, which once occurred almost immediately. In the presbyopic patient, these tasks can be performed only by the use of eyeglasses, contact lenses or after undergoing invasive surgery. A similar effect can be achieved with general miotic agents, such as pilocarpine (a non-selective muscarinic acetylcholine receptor agonist), carbachol (a non-selective muscarinic acetylcholine receptor agonist), and phospholine iodide (an acetylcholinesterase inhibitor). While these general miotic agents also create improved depth of focus via a pinhole effect induced by pupillary miosis (i.e. constriction), to the degree accommodation occurs, the pinhole effect only partially offsets the induced accommodative myopia for distance. These general miotic agents also cause substantial ciliary muscle spasms, which commonly induces discomfort that can be severe and long-lasting. In extreme cases, such ciliary muscle spasms can result in retinal detachment.

Even the use of aceclidine often result in unwanted ciliary spasms resulting in brow ache or generalized headaches. For example, the use of aceclidine alone resulted in significant brow ache. See U.S. Pat. Nos. 9,089,562, 9,833,441 and 9,314,427.

Efforts to overcome brow ache caused by unwanted ciliary spasms are based on the addition of a second ophthalmic drug. For example, U.S. Pat. No. 8,524,758 describes the use of pilocarpine with the non-steroidal anti-inflammatory, diclofenac, to reduce brow ache from ciliary spasm and increase the time in which the ciliary muscle contraction is regulated. U.S. Pat. Nos. 9,089,562, 9,833,441 and 9,314,427 each demonstrate that the addition of tropicamide to presbyopia treatments containing aceclidine result in a significant reduction in brow ache.

As described above, current efforts to achieve presbyopic treatment with miotic agents are limited by the ciliary induced brow ache that severely limit usefulness. Specifically, due to the need for lower concentrations that may somewhat reduce but not eliminate these bothersome side effects, only limited pupillary miosis occurs. This limited pupillary miosis limits depth of focus pinhole optic benefit to near vision and distance vision. Both distance and near optimal vision potential is compromised. Additionally, this limited pupillary miosis is sufficient only to partially offset the distance blur resulting from greater near point of focus. Further, only a few hours duration of this limited action can be achieved. The addition of a second ophthalmic drug to these miotic agents has been successful in overcoming brow ache caused by ciliary spasms. However, reduction in the number of drugs instilled in the eye to treat any single condition is advantageous. Thus, there is a need in the art for a treatment of presbyopia and other eye conditions that is non-invasive and convenient with minimal side effects. Specifically, there is a need for an ophthalmological compositions that will correct eye conditions such as presbyopia without inducing brow aches or headaches and without the need to add additional ophthalmic drugs.

### SUMMARY OF THE INVENTION

In certain embodiments, the present invention is directed to methods of reducing or eliminating brow ache when administering an ophthalmic drug comprising formulating the ophthalmic drug with a surfactant and a viscosity enhancer to create a composition having a viscosity of about 25 centipoise or less at a shear rate of 1/1000 per second at 25 degrees Celsius and a viscosity of about 70 centipoise at shear rate of 1 per second at 25 degrees Celsius and administering the composition to a subject in need thereof.

In certain other embodiments, the present invention is directed to methods of treating an eye condition selected from the group consisting presbyopia, myopia, hyperopia and astigmatism comprising administering a composition comprising a miotic agent, a surfactant and a viscosity enhancer to a subject in need thereof, wherein the composition has a viscosity of about 25 centipoise or less at a shear rate of 1/1000 per second at 25 degrees Celsius and a viscosity of about 70 centipoise or more, preferably about 150 centipoise or more and more preferably about 300 centipoise or more at shear rate of 1 per second at 25 degrees Celsius and wherein administration of the composition does not cause the subject to suffer from ciliary spasm induced brow ache or headache.

### DETAILED DESCRIPTION OF THE INVENTION

Applicant has unexpectedly discovered that the addition of a surfactant and a viscosity enhancer at concentrations that achieve a nonlinear viscosity for the composition reduce brow ache caused by ophthalmic drugs such as aceclidine.

In one embodiment, the present invention is directed to methods of reducing or eliminating brow ache when administering an ophthalmic drug comprising formulating the ophthalmic drug with a surfactant and a viscosity enhancer to create a composition having a viscosity of about 25 centipoise or less at a shear rate of 1/1000 per second at 25 degrees Celsius and a viscosity of about 70 centipoise at shear rate of 1 per second at 25 degrees Celsius and administering the composition to a subject in need thereof.

In another embodiment, the present invention is directed to methods of treating an eye condition selected from the group consisting of presbyopia, myopia, hyperopia and astigmatism comprising administering a composition comprising a miotic agent, a surfactant and a viscosity enhancer to a subject in need thereof, wherein the composition has a viscosity of about 25 centipoise or less at a shear rate of 1/1000 per second at 25 degrees Celsius and a viscosity of about 70 centipoise or more, preferably about 150 centipoise or more and more preferably about 300 centipoise or more at shear rate of 1 per second at 25 degrees Celsius and wherein administration of the composition does not cause the subject to suffer from ciliary spasm induced brow ache or headache.

Ophthalmic drugs that may benefit from the method of the present invention include miotic agents and phosphodiesterase inhibitors.

Phosphodiesterase inhibitors suitable for use in the present invention include, but are not limited to, adibendan, aminophylline, naminophylline dihydrate, namipizone, napremilast, narofylline, natizoram, nbefuraline, nbemarinone hydrochloride, nbemoradan, nbenafentrine, nbucladesine, nbuflomedil, nbuquineran, ncc-1088, ncarbazeran, catramilast, ncilomilast, cilostamide, ncilostazol, ncipamfylline, ncrisaborole, ndaxalipram, ndenbufylline, ndimabefylline, ndiniprofylline, ndipyridamole, ndoxofylline, ndrotaverine, ndyphylline, nenoximone, netamiphyllin, netofylline, nfilaminast, nflufylline, nfluprofylline, nfurafylline, nimazodan, nimazodan hydrochloride, ninamrinone, ninamrinone lactate, nisbufylline, nlirimilast, nlisofylline, nlomifylline, nmedorinone, nmetescufylline, nmidaxifylline, nmilrinone, nmilrinone lactate, nmotapizone, nnanterinone, nnestifylline, nnitraquazone, noglemilast, noglemilast sodium, nolprinone, noxagrelate, noxtriphylline, npapaverine, npapaverine hydrochloride, npapaverine sulfate, nparogrelil, npelrinone hydrochloride, npentifylline, npentoxifylline, nperbufylline, npiclamilast, npimefylline, npimobendan, npiroximone, nprinoxodan, nproxyphylline, npumafentrine, nquazinone, nquazodine, nrevamilast, nrevizinone, nroflumilast, nrolipram, nronomilast, nsaterinone, nsenazodan, nsiguazodan, ntetomilast, ntofimilast, ntrapidil, nvesnarinone, nzardaverine and combinations thereof,

Miotic agents suitable for use in the present invention include, but are not limited to, aceclidine, carbachol, pilocarpine, talsaclidine, sabcomeline, cevimeline, WAY-132983, AFB267B (NGX267), AC-42, AC-260584, 77-LH-28-1, and LY593039 or any pharmaceutically acceptable salts or esters thereof or combinations thereof. In a preferred embodiment, the miotic agent is selected from the group consisting of aceclidine, pilocarpine and carbachol. In a more preferred embodiment, the miotic agent is aceclidine.

Surfactants suitable for use in the present invention include nonionic, ionic and amphoteric (zwitterionic) surfactants. In a preferred embodiment, the surfactant used in the present invention are at a concentration above the critical micellar concentration for that surfactant.

Nonionic surfactants suitable for the present invention include cyclodextrins, polyoxyl alkyls, poloxamers, polysorbates or combinations thereof. Preferred embodiments include Poloxamer 108, Poloxamer 188, Poloxamer 407, Polysorbate 20, Polysorbate 80, ionically charged (e.g. anionic) beta-cyclodextrins with or without a butyrated salt (Captisol) 2-hydroxypropyl beta cyclodextrin (“HPβCD”), alpha cyclodextrins, gamma cyclodextrins, Polyoxyl 35 castor oil, and Polyoxyl 40 hydrogenated castor oil or combinations thereof. Further, substitution of other nonionic surfactants compatible with ophthalmological use allows for similar formulation advantages, which may included but is not limited to one or more of a nonionizing surfactant such as poloxamer, poloxamer 103, poloxamer 123, and poloxamer 124, poloxamer 407, poloxamer 188, and poloxamer 338, any poloxamer analogue or derivative, polysorbate, polysorbate 20, polysorbate 40, polysorbate 60, polysorbate 80, any polysorbate analogue or derivative, cyclodextrin, hydroxypropyl-β-cyclodextrin, hydroxypropyl-γ-cyclodextrin, randomly methylated β-cyclodextrin, β-cyclodextrin sulfobutyl ether, γ-cyclodextrin sulfobutyl ether or glucosyl-β-cyclodextrin, any cyclodextrin analogue or derivative, polyoxyethylene, polyoxypropylene glycol, an polysorbate analogue or derivative, polyoxyethylene hydrogenated castor oil 60, polyoxyethylene (200), polyoxypropylene glycol (70), polyoxyethylene hydrogenated castor oil, polyoxyethylene hydrogenated castor oil 60, polyoxyl, polyoxyl stearate, nonoxynol, octyphenol ethoxylates, nonyl phenol ethoxylates, capryols, lauroglycol, polyethylene glycol (“PEG”), Brij® 35, 78, 98, 700 (polyoxyethylene glycol alkyl ethers), glyceryl laurate, lauryl glucoside, decyl glucoside, or cetyl alcohol; or zwitterion surfactants such as palmitoyl carnitine, cocamide DEA, cocamide DEA derivatives cocamidopropyl betaine, or trimethyl glycine betaine, N-2(2-acetamido)-2-aminoethane sulfonic acid (ACES), N-2-acetamido iminodiacetic acid (ADA), N,N-bis(2-hydroxyethyl)-2-aminoethane sulfonic acid (BES), 2-[Bis-(2-hydroxyethyl)-amino]-2-hydroxymethyl-propane-1,3-diol (Bis-Tris), 3-cyclohexylamino-1-propane sulfonic acid (CAPS), 2-cyclohexylamino-1-ethane sulfonic acid (CHES), N,N-bis(2-hydroxyethyl)-3-amino-2-hydroxypropane sulfonic acid (DIPSO), 4-(2-hydroxyethyl)-1-piperazine propane sulfonic acid (EPPS), N-2-hydroxyethylpiperazine-N′-2-ethane sulfonic acid (HEPES), 2-(N-morpholino)-ethane sulfonic acid (MES), 4-(N-morpholino)-butane sulfonic acid (MOBS), 2-(N-morpholino)-propane sulfonic acid (MOPS), 3-morpholino-2-hydroxypropanesulfonic acid (MOPSO), 1,4-piperazine-bis-(ethane sulfonic acid) (PIPES), piperazine-N,N′-bis(2-hydroxypropane sulfonic acid) (POPSO), N-tris(hydroxymethyl)methyl-2-aminopropane sulfonic acid (TAPS), N-[tris(hydroxymethyl)methyl]-3-amino-2-hydroxypropane sulfonic acid (TAPSO), N-tris(hydroxymethyl) methyl-2-aminoethane sulfonic acid (TES), 2-Amino-2-hydroxymethyl-propane-1,3-diol (Tris), tyloxapol, Solulan™ C-24 (2-[[10,13-dimethyl-17-(6-methylheptan-2-yl)-2,3,4,7,8,9,11,12,14,15,16,17-dodecahydro-1H-cyclopenta[a]phenanthren-3-yl]oxy]ethanol) and Span® 20-80 (sorbitan monolaurate, sorbitan monopalmitate, sorbitan monostearate, and sorbitan monooleate). In other embodiments the addition of polysorbate 80 is preferred. In addition to the above nonionic surfactants any nonionic surfactant is suitable for use in the present invention as long as the concentration of the nonionic surfactant is such that it is above the critical micellar concentration for that non-ionic surfactant. Preferably, the nonionic surfactants used in the present invention achieve submicron diameter micelles, more preferably less than 200 nanometers and more preferably less than 150 nanometers in diameter.

Ionic surfactants suitable for use in the present invention include, but are not limited to, anionic surfactants and cationic surfactants. Anionic surfactants suitable for use in the present invention include, but are not limited to, ammonium lauryl sulfate, dioctyl sodium sulfosuccinate, sodium laureth sulfate, linear alkylbenzene sulfonate, sodium dodecyl sulfate, perfluorooctanesulfonate, sodium lauryl sarcosinate, sodium myreth sulfate, sodium pareth sulfate, sodium stearate, lignosulfonate, sodium lauryl sulfate, a olefin sulfonate, ammonium laureth sulfate and sodium ester lauryl sulfate. Cationic surfactants suitable for use in the present invention include, but are not limited to benzalkonium chloride, benzethonium chloride, methylbenzethonium chloride, cetylpyridinium chloride, alkyl-dimethyl dichlorobenzene ammonium chloride, dequalinium chloride, phenamylinium chloride, cetyl trimethylammonium bromide, cetyl trimethylammonium chloride, cetrimonium bromide and cethexonium bromide.

Amphoteric (zwitterionic) surfactants are any surfactant simultaneously carrying an anionic charge and a cationic charge. Amphoteric surfactants suitable for use in the present invention include, but are not limited to, alkyl or alkenyl-amphoacetates or ampho-diacetates, alkylampho-propionates or -dipropionates, alkyl amphohydroxypropyl sultaines wherein the alkyl groups contain 8 to 24 carbon atoms such as coco or lauryl.

Surfactants may be used in the present invention at a concentration above their critical micellar concentration. The critical micellar concentration for any particular surfactant may be calculated by a person of skill in the art. In a preferred embodiment, the concentration of surfactants in the present invention is from about 1.5% to about 7% w/v, more preferably from about 1.5% to about 5% w/v.

Ophthalmological in situ gels which may be substituted for or added in addition to one or more surfactants include but are not limited to gelatin, carbomers of various molecular weights including carbomer 934 P and 974 P, xanthan gums, alginic acid (alginate), guar gums, locust bean gum, chitosan, pectins and other gelling agents well known to experts in the art.

In other preferred embodiments, the surfactant is polysorbate 80 at a concentration from about 0.5% to about 10% w/v, more preferably from about 1% to about 7% w/v and even more preferably from about 1.5% to about 5% w/v, yet more preferably from about 2.5% to about 4% w/v, yet more preferably from about 3% to about 4% and most preferably at about 2.5% or 2.75% or 3% or 4% or 5% w/v.

Viscosity agents suitable for the present invention include, but are not limited to gums such as guar gum, hydroxypropyl-guar (“hp-guar”), and xanthan gum, alginate, chitosan, gelrite, hyaluronic acid, dextran, Carbopol® (polyacrylic acid or carbomer) including Carbopol® 900 series including Carbopol® 940 (carbomer 940), Carbopol® 910 (carbomer 910) and Carbopol® 934 (carbomer 934), cellulose derivatives such as carboxymethyl cellulose (“CMC”), methylcellulose, methyl cellulose 4000, hydroxymethyl cellulose, hydroxypropyl cellulose, hydroxypropylmethyl cellulose, hydroxyl propyl methyl cellulose 2906, carboxypropylmethyl cellulose, hydroxypropylethyl cellulose, and hydroxyethyl cellulose, polyethylene glycol, polyvinyl alcohol, polyvinyl pyrrolidone, gellan, carrageenan, alginic acid, carboxyvinyl polymer or combinations thereof.

Viscosity agents may be used in the present invention at a concentration necessary to achieve a viscosity of about 0.5 centipoise or less at a shear rate of 1/1000 per second at 25 degrees Celsius and a viscosity of about 70 centipoise or more, preferably 150 centipoise or more and preferably 300 centipoise or more at shear rate of 1 per second at 25 degrees Celsius when combined with a surfactant above its critical micellar concentration.

In certain other embodiments, the compositions of the present invention has a viscosity from about 75 to about 1,000 centipoise at a shear rate of 0 at 25 degrees Celsius, preferably from about 75 to about 500 centipoise at a shear rate of 0 at 25 degrees Celsius.

In a preferred embodiment the viscosity agent will have an equilibration viscosity less than 100 cps, preferably from about 15 to about 35 cps, and most preferably at about 30 cps.

In a preferred embodiment the viscosity agent is Carbopol® 940 (carbomer 940) at a concentration from about 0.05% to about 1.5% w/v, preferably from about 0.09% to about 1.0% w/v, more preferably at 0.09%, 0.25%, 0.5%, 0.75%, 0.9% or 1.0% w/v.

In another preferred embodiment, the viscosity agent is carboxymethyl cellulose at a concentration from about 1% to about 2% w/v, more preferably from 1.35% to about 1.45% w/v and most preferably 1.42% w/v or 1.40% w/v.

In another preferred embodiment, the viscosity agent is hydroxypropylmethyl cellulose at a concentration from about 0.5% to about 1.75%, and more preferably about 0.75% or 1.5%, still more preferably from about 1.0% to about 1.5%, and most preferably at about 1.25%.

Compositions of the present invention may further comprise cryoprotectants, polyols, bulking agents, solubilizers, antioxidants, tonicity adjustors, preservatives, Cryoprotectants are compounds that either prevent freezing or prevent damage to compounds during freezing. As used herein, the term “cryoprotectant” or “cryoprotectants” include lyoprotectants.

Cryoprotectants suitable for use in the subject invention include, but are not limited to, a polyol, a sugar, an alcohol, a lower alkanol, a lipophilic solvent, a hydrophilic solvent, a bulking agent, a solubilizer, a surfactant, an antioxidant, a cyclodextrin, a maltodextrin, colloidal silicon dioxide, polyvinyl alcohol, glycine, 2-methyl-2,4-pentanediol, cellobiose, gelatin, polyethylene glycol (PEG), dimethyl sulfoxide (DMSO), formamide, antifreeze protein 752 or a combination thereof.

As used herein the term “polyol” refers to compounds with multiple hydroxyl functional groups available for organic reactions such as monomeric polyols such as glycerin, pentaerythritol, ethylene glycol and sucrose. Further, polyols may refer to polymeric polyols including glycerin, pentaerythritol, ethylene glycol and sucrose reacted with propylene oxide or ethylene oxide. In a preferred embodiment, polyols are selected from the group consisting of mannitol, glycerol, erythritol, lactitol, xylitol, sorbitol, isosorbide, ethylene glycol, propylene glycol, maltitol, threitol, arabitol and ribitol. In a more preferred embodiment, the polyol is mannitol.

Sugars suitable for use in the present invention as cryoprotectants include, but are not limited to, glucose, sucrose, trehalose, lactose, maltose, fructose and dextran.

In another preferred embodiment, alcohols include, but are not limited to, methanol.

In one embodiment, the present invention individually excludes each cryoprotectant from the definition of cryoprotectant.

Cryoprotectants may be at present in compositions of the present invention at a concentration from about 0.1% to about 99% w/v, preferably from about 1% to about 50% w/v, more preferably from about 1% to about 10% w/v.

As used herein “lower alkanols” include C1-C6 alkanols. Lower alkanols, suitable for use in the present invention include, but are not limited to, amyl alcohol, butanol, sec-butanol, t-butyl alcohol, n-butyl alcohol, ethanol, isobutanol, methanol. isopropanol and propanol.

Bulking agents suitable for use in the present invention include, but are not limited to, saccharide, polyvinylpyrrolidone, cyclodextrin and trehalose.

Solubilizers suitable for use in the present invention include, but are not limited to, cyclic amide, gentisic acid and cyclodextrins.

In a preferred embodiment, antioxidants suitable for use in the present invention include, but are not limited to, bisulfite, ascorbic acid, disodium- or tetrasodium ethylenediaminetetraacetic acid, citrate, butylated hydroxyanisole (“BHA”), butylated hydroxytoluene (“BHT”), a sulfoxylate, propyl gallate, an amino acid containing a thio group, and a thiol. In a preferred embodiment the antioxidant is disodium ethylenediaminetetraacetic acid at a concentration from about 0.005% to about 0.50% w/v, citrate at a concentration from about 0.01% to about 0.3% w/w, dicalcium diethylenetriamine pentaacetic acid (“Ca2DTPA”) at a concentration from about 0.001% to about 0.2% w/v, preferably about 0.01% w/v Ca2DTPA which can be formulated by adding 0.0084% w/v Ca(OH)2 and 0.0032% w/v pentetic acid to the formulation and mixing slowly. Further combinations of antioxidants can be used. Other antioxidants that can be used with the present invention include those well known to experts in the art such as ethylenediaminetetraacetic acid at a concentration from about 0.0001% to about 0.015% w/v.

A tonicity adjustor can be, without limitation, a salt such as sodium chloride (“NaCl”), potassium chloride, mannitol or glycerin, or another pharmaceutically or ophthalmologically acceptable tonicity adjustor. In certain embodiments the tonicity adjustor is 0.037% w/v NaCl,

Preservatives that can be used with the present invention include, but are not limited to, benzalkonium chloride (“BAK”), sorbic acid, oxychloro complex, citric acid, chlorobutanol, thimerosal, phenylmercuric acetate, disodium ethylenediaminetetraacetic acid, phenylmercuric nitrate, perborate or benzyl alcohol. In a preferred embodiment the preservative is BAK, sorbic acid, oxychloro complex or a combination thereof. In a yet more preferred embodiment BAK is at a concentration of about 0.001% to about 1.0% w/v, more preferably at a concentration of about 0.007%, 0.01% or 0.02% w/v. In another preferred embodiment the preservative is perborate at a concentration of 0.01% to about 1.0% w/v, more preferably at a concentration of about 0.02% w/v.

Various buffers and means for adjusting pH can be used to prepare ophthalmological compositions of the invention. Such buffers include, but are not limited to, acetate buffers, citrate buffers, phosphate buffers and borate buffers. It is understood that acids or bases can be used to adjust the pH of the composition as needed, preferably of 1 to 10 mM concentration, and more preferably about 3 mM or 5 mM. In a preferred embodiment the pH is from about 4.0 to about 8.0, in a more preferred embodiment the pH is from about 5.0 to about 7.0.

As used herein, the terms “reduce” or “reducing” refer to decreasing the duration or intensity.

As used herein, the term “composition” is intended to encompass a product comprising the specified ingredients in the specified amounts, as well as any product which results, directly or indirectly, from a combination of the specified ingredients in the specified amounts.

As used herein, all numerical values relating to amounts, weights, and the like, that are defined as “about” each particular value is plus or minus 10%. For example, the phrase “about 5% w/v” is to be understood as “4.5% to 5.5% w/v.” Therefore, amounts within 10% of the claimed value are encompassed by the scope of the claims.

As used herein “% w/v” refers to the percent weight of the total composition.

As used herein the term “subject” refers but is not limited to a person or other animal.

The term “aceclidine” encompasses its salts, esters, analogues, prodrugs and derivatives including, but not limited to, aceclidine as a racemic mixture, aceclidine (+) enantiomer, aceclidine (−) enantiomer, aceclidine analogues, including, but not limited to, highly M1 selective 1,2,5 thiadiazole substituted analogues like those disclosed in Ward. J. S. et al., 1,2,5-Thiadiazole analogues of aceclidine as potent ml muscarinic agonists, J Med Chem, 1998, Jan. 29, 41(3), 379-392 and aceclidine prodrugs including but not limited to carbamate esters.

The term “pharmaceutically acceptable” describes a material that is not biologically or otherwise undesirable (i.e. without causing an unacceptable level of undesirable biological effects or interacting in a deleterious manner).

As used herein, the term “pharmaceutically effective amount” refers to an amount sufficient to affect a desired biological effect, such as a beneficial result, including, without limitation, prevention, diminution, amelioration or elimination of signs or symptoms of a disease or disorder. Thus, the total amount of each active component of the pharmaceutical composition or method is sufficient to show a meaningful subject benefit. Thus, a “pharmaceutically effective amount” will depend upon the context in which it is being administered. A pharmaceutically effective amount may be administered in one or more prophylactic or therapeutic administrations.

As used herein “salts” refers to those salts which retain the biological effectiveness and properties of the parent compounds and which are not biologically or otherwise harmful at the dosage administered. Salts of the compounds of the present inventions may be prepared from inorganic or organic acids or bases.

The compounds of the present invention can be used in the form of pharmaceutically acceptable salts derived from inorganic or organic acids or bases. The phrase “pharmaceutically acceptable salt” means those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, 1977, 66: 1 et seq.

The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, hyaluronic acid, malic acid, sulphuric acid and phosphoric acid and such organic acids as oxalic acid, malic acid, maleic acid, methanosulfonic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylammonium, dimethylammonium, trimethylammonium, triethylammonium, diethylammonium, and ethylammonium among others. Other representative organic amines useful for the formation of base addition salts include ethylenediamine, ethanolamine, diethanolamine, piperidine, piperazine and the like.

The term “ester” as used herein is represented by the formula —OC(O)A1 or —C(O)OA1, where A1 can be alkyl, cycloalkyl, alkenyl, cycloalkenyl, alkynyl, cycloalkynyl, aryl, a heteroaryl group or another suitable substituent.

In a preferred embodiment, the present invention is directed to methods of treating an eye condition selected from the group consisting of presbyopia, myopia, hyperopia and astigmatism comprising, consisting essentially of or consisting of administering a composition comprising aceclidine, a surfactant and a viscosity enhancer to a subject in need thereof, wherein the composition has a viscosity of about 25 centipoise or less at a shear rate of 1/1000 per second at 25 degrees Celsius and a viscosity of about 70 centipoise or more, preferably about 150 centipoise or more and more preferably about 300 centipoise or more at shear rate of 1 per second at 25 degrees Celsius and wherein administration of the composition does not cause the subject to suffer from ciliary spasm induced brow ache or headache.

In a more preferred embodiment, the present invention is directed to methods of treating an eye condition selected from the group consisting of presbyopia, myopia, hyperopia and astigmatism comprising, consisting essentially of or consisting of administering a composition comprising aceclidine, polysorbate 80 and carboxymethyl cellulose to a subject in need thereof, wherein the composition has a viscosity of about 25 centipoise or less at a shear rate of 1/1000 per second at 25 degrees Celsius and a viscosity of about 70 centipoise or more, preferably about 150 centipoise or more and more preferably about 300 centipoise or more at shear rate of 1 per second at 25 degrees Celsius and wherein administration of the composition does not cause the subject to suffer from ciliary spasm induced brow ache or headache.

In another embodiment, the methods of the present invention comprise compositions wherein the composition comprises a miotic agent as the sole active ingredient.

In another embodiment, the methods of the present invention comprise compositions that do not contain a cycloplegic agent, for example tropicamide.

In another embodiment, the methods of the present invention comprise compositions that do not contain a polyol, for example mannitol.

The following Example is provided solely for illustrative purposes and is not meant to limit the invention in any way.

### Example (Prophetic)

TABLE 1

| Formulation | #1 | #1A | #1B | #1C | #2 | #2A | #2B | #2C |
|---|---|---|---|---|---|---|---|---|
| Aceclidine | 1.75% | 1.75% | 1.75% | 1.75% | — | — | — | — |
| Pilocarpine | — | — | — | — | 1.1% | 1.1% | 1.1% | 1.1% |
| Carbachol | — | — | — | — | — | — | — | — |
| Polysorbate 80 | — | 4.0% | — | 4.0% | — | 4.0% | — | 4.0% |
| Hydroxypropylmethyl | — | — | 1.2% | 1.2% | — | — | 1.2% | 1.2% |
| Cellulose | | | | | | | | |
| Water | Q.S. | Q.S. | Q.S. | Q.S. | Q.S. | Q.S. | Q.S. | Q.S. |
| Viscosity at 1/s Shear | 1 | <1 | >250 | 350 | 1 | <1 | >250 | 350 |
| Rate (cps) | | | | | | | | |
| Viscosity at 1/1000/s | 1 | <1 | <250 | 25 | 1 | <1 | >250 | <25 |
| Shear Rate (cps) | | | | | | | | |
| Ciliary Spasm | 2 | 2 | 2 | 0.5 | 4 | 4 | 4 | 0.5 |

| Formulation | #3 | #3A | #3B | #3C |
|---|---|---|---|---|
| Aceclidine | | | | |
| Pilocarpine | | | | |
| Carbachol | 0.01% | 0.01% | 0.01% | 0.01% |
| Polysorbate 80 | — | 4.0% | — | 4.0% |
| Hydroxypropylmethyl | — | — | 1.2% | 1.2% |
| Cellulose | | | | |
| Water | Q.S. | Q.S. | Q.S. | Q.S. |
| Viscosity at 1/s Shear | 1 | ≤1 | >250 | 320 |
| Rate (cps) | | | | |
| Viscosity at 1/1000/s | 1 | ≤1 | >250 | <25 |
| Shear Rate (cps) | | | | |
| Ciliary Spasm | 4 | 4 | 4 | 0.5 |

### Method

Formulations of Table 1 were each separately instilled in the eye of a presbyopic subject with a 2-week washout period between instillations. Patient was assessed for brow ache induced ciliary spasms based on the following criteria: 0=no discomfort; 0.5=slight sting; 1=noticeable squeeze/discomfort; 2=pain for less than 30 minutes; 3=pain for 1 hour or more; and 4=severe to intolerable pain.

### Results

As seen in Table 1, the addition of a surfactant and a viscosity enhancer at concentrations providing a nonlinear viscosity to the composition result in significant reduction in pain caused by ciliary spasms.

What is claimed is:

1. A method of reducing or eliminating brow ache when administering an ophthalmic drug comprising formulating the ophthalmic drug with a surfactant and a viscosity enhancer to create a composition having a viscosity selected from the group consisting of from about 70 to about 500 centipoise at shear rate of 0 at 25 degrees Celsius, about 25 centipoise or less at a shear rate of 1/1000 per second at 25 degrees Celsius, a viscosity of about 70 centipoise at shear rate of 1 per second at 25 degrees Celsius and combinations thereof and administering the composition to a subject in need thereof.

2. The method of claim 1, wherein the composition has a viscosity of about 25 centipoise or less at a shear rate of 1/1000 per second at 25 degrees Celsius and a viscosity of about 70 centipoise or more at shear rate of 1 per second at 25 degrees Celsius.

3. The method of claim 1, wherein the brow ache is induced by ciliary spasms.

4. The method of claim 1, wherein the viscosity at shear rate of 1 per second at 25 degrees Celsius is about 150 centipoise or more.

5. The method of claim 1, wherein the viscosity at shear rate of 1 per second at 25 degrees Celsius is about 300 centipoise or more.

6. The method of claim 1, wherein the ophthalmic drug is selected from the group consisting of aceclidine, pilocarpine, talsaclidine, sabcomeline, cevimeline, WAY-132983, AFB267B (NGX267), AC-42, AC-260584, 77-LH-28-1, and LY593039, a pharmaceutically acceptable salt thereof, an ester thereof and a combination thereof.

7. The method of claim 6, wherein the ophthalmic drug is selected from the group consisting of aceclidine, pilocarpine, a pharmaceutically acceptable salt thereof, an ester thereof and a combination thereof.

8. The method of claim 1, wherein the ophthalmic drug is aceclidine a pharmaceutically acceptable salt thereof or an ester thereof.

9. The method of claim 1, wherein the surfactant is selected from the group consisting of polysorbates, poloxamers, polyoxyl alkyls, cyclodextrins, ammonium lauryl sulfate, dioctyl sodium sulfosuccinate, sodium laureth sulfate, linear alkylbenzene sulfonate, sodium dodecyl sulfate, perfluorooctanesulfonate, sodium lauryl sarcosinate, sodium myreth sulfate, sodium pareth sulfate, sodium stearate, lignosulfonate, sodium lauryl sulfate, a olefin sulfonate, ammonium laureth sulfate, sodium ester lauryl sulfate, benzalkonium chloride, benzethonium chloride, methylbenzethonium chloride, cetylpyridinium chloride, alkyl-dimethyl dichlorobenzene ammonium chloride, dequalinium chloride, phenamylinium chloride, cetyl trimethylammonium bromide, cetyl trimethylammonium chloride, cetrimonium bromide, cethexonium bromide, alkyl-amphoacetates, alkenyl-amphoacetates, alkyl-amphodiacetates, alkenyl-amphodiacetates, alkylamphopropionates, alkylamphodipropionates, alkyl amphohydroxypropyl sultaines and combinations thereof.

10. The method of claim 1, wherein the viscosity enhancer is selected from the group consisting of gums, cellulose derivatives, polyethylene glycol, polyvinyl alcohol, polyvinyl pyrrolidone, gellan, carrageenan, alginic acid, carboxyvinyl polymer and combinations thereof.

11. The method of claim 1, wherein the surfactant is at a concentration from about 1.5% to about 7.0% w/v, wherein w/v denotes weight by total volume of the composition.

12. The method of claim 1, wherein the composition further comprises one or more excipients selected from the group consisting of cryoprotectants, polyols, bulking agents, solubilizers, antioxidants, tonicity adjustors and preservatives.

13. The method of claim 1, wherein the ophthalmic drug is the sole active ingredient in the composition.

14. The method of claim 1, wherein the composition does not contain a polyol.

15. The method of claim 1, wherein the composition does not contain mannitol.

16. The method of claim 1, wherein the composition does not contain a cycloplegic agent.

17. The method of claim 1, wherein the composition does not contain tropicamide.

18. A method of treating an eye condition selected from the group consisting of presbyopia, myopia, hyperopia and astigmatism comprising administering a composition comprising a miotic agent, a surfactant and a viscosity enhancer to a subject in need thereof, wherein the composition has a viscosity selected from the group consisting of from about 70 to about 500 centipoise at shear rate of 0 at 25 degrees Celsius, about 25 centipoise or less at a shear rate of 1/1000 per second at 25 degrees Celsius, a viscosity of about 70 centipoise or more at shear rate of 1 per second at 25 degrees Celsius and combinations thereof and wherein administration of the composition does not cause the subject to suffer from ciliary spasm induced brow ache or headache.

19. The method of claim 18, wherein the miotic agent is selected from the group consisting of aceclidine, pilocarpine, talsaclidine, sabcomeline, cevimeline, WAY-132983, AFB267B (NGX267), AC-42, AC-260584, 77-LH-28-1, and LY593039, a pharmaceutically acceptable salt thereof, an ester thereof and a combination thereof.

20. The method of claim 18, wherein the miotic agent is the sole active ingredient in the composition.

21. The method of claim 18, wherein the composition does not contain a polyol.

22. The method of claim 18, wherein the composition does not contain mannitol.

23. The method of claim 18, wherein the composition does not contain a cycloplegic agent.

24. The method of claim 18, wherein the composition does not contain tropicamide.
